(19) United States
(12) Patent Application Publication
(10) Pub. No.: US 2023/0301672 A1
(43) Pub. Date: Sep. 28, 2023

(54) ASPIRATION CATHETER INCLUDING A LUBRICIOUS INNER LINER AND FLEXIBLE DISTAL TIP

(71) Applicant: Deepin Technologies, LLC, San Jose, CA (US)

(72) Inventor: Jon Schabert, San Jose, CA (US)

(73) Assignee: Deepin Technologies, LLC, San Jose, CA (US)

(21) Appl. No.: 18/174,624

(22) Filed: Feb. 26, 2023

Related U.S. Application Data
(60) Provisional application No. 63/322,508, filed on Mar. 22, 2022.

(51) Int. Cl. A61B 17/22

(52) U.S. Cl. CPC A61B 17/22; A61B 2017/22079; A61M 2025/0681

(57) ABSTRACT

An aspiration catheter includes an elongate tubular body having a proximal end portion and a distal end portion. The elongate tubular body comprises an inner liner and one or more outer layers. The inner liner comprises a first surface defining a lumen of the elongate tubular body and a second surface. The one or more outer layers are disposed over the second surface of the inner liner. In an embodiment, the inner liner has a first thickness at the proximal end portion of the elongate tubular body and a second thickness at the distal end portion of the elongate tubular body smaller than the first thickness. In another embodiment, the inner liner is provided with plural voids between the first surface and the second surface of the inner liner.

### CROSS-REFERENCE TO RELATED APPLICATIONS

This application claims priority to U.S. provisional patent application No. 63/322,508 filed Mar. 22, 2022 entitled “Aspiration Catheter with Lubricious Inner Liner with an Improved Flexible Distal Tip for Improved Trackability,” the disclosure of which is hereby incorporated by reference in its entirety.

### TECHNICAL FIELD

This application relates generally to medical catheters and methods of making and using medical catheters. In particular, various embodiments of an aspiration catheter useful in thrombectomy and other medical procedures are described.

### BACKGROUND

Stroke is one of the leading causes of death worldwide with an annual mortality rate of more than five million. In the United State, every year about 800, 000 people have a stroke. With all the medical advancement, over 17% of the patients still die. Patients who survive stroke may end up with long-term disability, creating immense economic and social consequences.

An ischemic stroke occurs when clots or other occlusions block the blood vessels to the brain. A hemorrhagic stroke happens when an artery in the brain ruptures. In either case, parts of the brain become damaged or die, causing brain damage, long-term disability, or death. Ischemic stroke is the more common type of stroke.

Various medical procedures are known and have been used in treating stroke. For example, in a thrombectomy procedure for treating ischemic stroke, an aspiration catheter may be used to remove occlusions such as clots from blood vessels. While significant advancement in aspiration thrombectomy has been achieved, there remains a general need for improvement. For example, it would be desirable to provide an aspiration catheter with improved flexibility and trackability to navigate the complex, tortuous cerebral anatomy in performing various medical procedures including removing occlusions or clots to treat stroke.

### SUMMARY

In one aspect, embodiments of the disclosure feature a catheter. In general, an embodiment of the catheter comprises an elongate tubular body having a proximal end portion and a distal end portion. The elongate tubular body comprises an inner liner and one or more outer layers. The inner liner comprises a first surface defining a lumen of the elongate tubular body and a second surface. The one or more outer layers are disposed over the second surface of the inner liner. The inner liner has a first thickness at the proximal end portion of the elongate tubular body and a second thickness at the distal end portion of the elongate tubular body, where the second thickness is smaller than the first thickness.

In various embodiments of the aspect, the second surface of the inner liner defines a first outer diameter at the proximal end portion of the elongate tubular body and a second outer diameter at the distal end portion of the elongate tubular body, wherein the second outer diameter is smaller than the first outer diameter.

In an embodiment, the first outer diameter defined by the second surface of the inner liner at the proximal end portion is substantially constant, the second outer diameter defined by the second surface of the inner liner at the distal end portion is substantially constant, and the second surface of the inner liner comprises a transition section having a tapering function from the first outer diameter to the second outer diameter.

In another embodiment, the first outer diameter defined by the second surface of the inner liner at the proximal end portion is substantially constant, the second outer diameter defined by the second surface of the inner liner at the distal end portion is substantially constant, and the second surface of the inner liner comprises a transition section having a step function from the first outer diameter to the second outer diameter.

In a further embodiment, the first surface of the inner liner defines a diameter of the lumen substantially constant between the proximal end portion and the distal end portion of the elongate tubular body.

In various embodiments of the aspect, the inner liner is constructed from a material comprising polytetrafluoroethylene (PTFE).

In various embodiments of the aspect, the inner liner is provided with plural voids between the first surface and the second surface of the inner liner.

In some embodiments, the plural voids are located at the distal end portion of the elongate tubular body. In some embodiments, the plural voids are located throughout the entire length of the elongate tubular body from the proximal end portion to the distal end portion.

In another aspect, embodiments of the disclosure feature a catheter. In general, an embodiment of the catheter comprises an elongate tubular body having a proximal end portion and a distal end portion. The elongate tubular body comprises an inner liner and one or more outer layers. The inner liner comprises a first surface defining a lumen of the elongate tubular body and a second surface. The one or more outer layers are disposed over the second surface of the inner liner. The inner liner is provided with plural voids between the first surface and the second surface of the inner liner.

In various embodiments of the aspect, the plural voids are located at the distal end portion of the elongate tubular body.

In various embodiments of the aspect, the plural voids are located throughout the entire length of the elongate tubular body from the proximal end portion to the distal end portion.

In various embodiments of the aspect, the inner liner comprises a first thickness at the proximal end portion of the elongate tubular body and a second thickness at the distal end portion of the elongate tubular body. The second thickness is smaller than the first thickness.

In various embodiments of the aspect, the second surface of the inner liner defines a first outer diameter at the proximal end portion of the elongate tubular body and a second outer diameter at the distal end portion of the elongate tubular body. The second outer diameter is smaller than the first outer diameter. The first outer diameter defined by the second surface of the inner liner at the proximal end portion can be substantially constant, and the second outer diameter defined by the second surface of the inner liner at the distal end portion can be substantially constant. In an embodiment, the second surface of the inner liner further comprises a transition section having a tapering function from the first outer diameter to the second outer diameter. In another embodiment, the second surface of the inner liner further comprises a transition section having a step function from the first outer diameter to the second outer diameter.

In various embodiments of the aspect, the inner liner is constructed from a material comprising polytetrafluoroethylene (PTFE).

In various embodiments of the aspect, the elongate tubular body comprises a length between the proximal end portion and the distal end portion, and the inner liner extends the length of the elongate tubular body and comprises a substantially constant thickness along the length. The plural voids can be located at the distal end portion of the elongate tubular body. The plural voids can also be located throughout the entire length of the elongate tubular body.

This Summary is provided to introduce selected aspects and embodiments of this disclosure in a simplified form and is not intended to identify key features or essential characteristics of the claimed subject matter, nor is it intended to be used as an aid in determining the scope of the claimed subject matter. The selected aspects and embodiments are presented merely to provide the reader with a brief summary of certain forms the invention might take and are not intended to limit the scope of the invention. Other aspects and embodiments of the disclosure are described in the section of Detailed Description.

These and various other aspects, embodiments, features, and advantages of the disclosure will become better understood upon reading of the following detailed description in conjunction with the accompanying drawings.

### BRIEF DESCRIPTION OF THE DRAWINGS

FIG. 1 is a simplified illustration of an example aspiration system according to embodiments of the disclosure.

FIG. 2 is an enlarged cross-sectional view of a portion of an example aspiration catheter according to embodiments of the disclosure.

FIGS. 3A and 3B are enlarged cross-sectional views of a portion of an example inner liner having a variable thickness according to embodiments of the disclosure.

FIGS. 4A and 4B are enlarged cross-sectional views of a portion of an example inner liner provided with voids or bubbles according to embodiments of the disclosure.

FIGS. 5A and 5B are enlarged cross-sectional views of a portion of an example inner liner provided with voids or bubbles according to alternative embodiments of the disclosure.

FIG. 6 is a simplified illustration of an example aspiration system of the disclosure used in a procedure for removing an occlusion within a blood vessel in the cerebral anatomy of a patient.

### DETAILED DESCRIPTION OF EMBODIMENTS

With reference to the figures, various embodiments of an aspiration catheter, system, and method will now be described. The figures are intended to facilitate illustration and are not necessarily drawn to scale. Certain specific details may be set forth in the figures and description to provide a thorough understanding of the disclosure. It will be apparent to one of ordinary skill in the art that some of these specific details may not be employed to practice embodiments of the disclosure. In other instances, structures, materials, components, systems, and/or operations often associated with intravascular procedures are not shown or described in detail to avoid unnecessarily obscuring embodiments of the disclosure.

Embodiments of the disclosure provide an aspiration catheter including an inner liner constructed to improve the flexibility and trackability of the catheter desirable for performing various medical procedures, including aspiration thrombectomy in the cerebral anatomy. The inner liner of the aspiration catheter can be constructed with a smaller thickness at the tip or distal section of the catheter to reduce stiffness. This can be achieved by selectively removing a portion of the inner liner at the tip or distal section via mechanical, thermal, or chemical means, and returning to an original or initial thickness of the remaining section via a step or tapering function. The thinning of the inner liner can also be achieved by selectively depositing layers on a mandrel to build up the inner liner and at some point, discontinuing deposition of the material at the distal end or section of the inner liner. Alternatively, or additionally, small voids or bubbles can be created in the inner liner to increase the flexibility of the catheter. The bubbles can be formed in the inner liner throughout the entire length of the catheter, or just through a distal end section. By forming bubbles in the inner liner, the liner material is replaced with empty space, resulting in the same effect as thinning the inner liner and thus improving the flexibility of the catheter. Generally, the inner liner of a catheter is made of a stiff material. Reducing the thickness of the inner liner at the distal end of a catheter, and/or, creating voids or bubbles in the inner liner can reduce the stiffness of the liner, thereby improving the flexibility and trackability of the overall catheter. The improved flexibility and trackability can help the user navigate the catheter through the complex, tortuous vasculature at the cerebral anatomy, thereby facilitating medical procedures including aspiration thrombectomy to treat stroke.

It should be pointed that while the term “aspiration” is used in describing various embodiments of the disclosure, the catheter, system, and method described herein can be used in medical procedures other than or in addition to aspiration. For example, the catheter can be used to deliver therapeutical agents, fluids or medical devices to a treatment site in a vasculature, or as a support catheter to provide a conduit that facilitates and guides delivery of other devices such as interventional devices. Further, it should be appreciated that while embodiments of the disclosure may be described in conjunction with an aspiration thrombectomy procedure in the cerebral anatomy, the catheter, system, and method described herein can be configured to access, deliver agents or devices, or perform procedures in other anatomies such as peripheral and coronary anatomies.

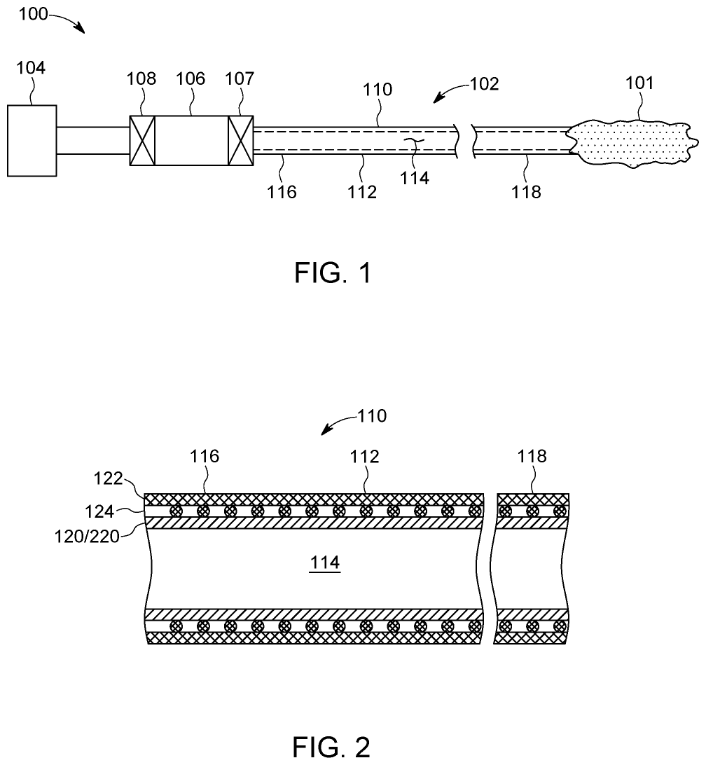

FIG. 1 depicts an example aspiration system 100 according to embodiments of the disclosure. The aspiration system 100 can be used to remove occlusions such as a clot 101 within the vasculature at the cerebral anatomy or other treatment sites. As shown, the aspiration system 100 in general comprises a catheter device 102, a vacuum source 104, and an interface unit or hub 106 operably connecting the catheter device 102 with the vacuum source 104. The hub 106 may be provided with one or more ports or ducts for connecting with the catheter device 102, the vacuum source 104, and other devices or fluid sources via tubing and/or connectors. The catheter device 102 may be connected to the hub 106 via a valve 107 such as a rotary hemostatic valve (RHV). An RHV allows for introduction of a device into the vasculature while preventing or minimizing blood loss and preventing air introduction into the vasculature. The vacuum source 104 may be provided by a vacuum pump, a syringe, or other suitable vacuum sources. The vacuum source 104 may be connected to the hub 106 via a valve 108 such as an RHV. Therefore, in operation, the catheter device 102 may access to a treatment site and operate to aspirate or remove an occlusion 101 upon application of a negative pressure provided by the vacuum source 104. The negative pressure provided by the vacuum source 104 may be constant or pulsatile. While not shown in FIG. 1, the aspiration system 100 may also include a fluid source operably connected to the hub 106 via a connection port. The fluid source may provide saline, blood, or other fluids which may be needed for performing the procedure.

The catheter device 102 may include an aspiration catheter 110 configured to access and aspirate an occlusion or clot 101 from a treatment site. The aspiration catheter 110 comprises an elongate tubular body 112 defining a working lumen 114 extending between a proximal end portion 116 and a distal end portion 118 of the elongate tubular body 112. The proximal end portion 116 of the aspiration catheter 110 may be coupled to the hub 106, forming a fluidic communication between the working lumen 114 and the vacuum source 104. As will be described in greater detailed below, the aspiration catheter 110 of the disclosure has a higher degree of flexibility and trackability, and is capable of providing an access to locations even through the extreme tortuosity at the cerebral anatomy. As such, the aspiration catheter 110 of the disclosure allows for delivering aspirational forces to a treatment site of extreme tortuosity, and/or, for delivering interventional devices such as a stent, stent retriever, flow diverter, or other devices to a treatment site of extreme tortuosity.

While not shown in FIG. 1, in some embodiments of the disclosure, the catheter device 102 may also include an introducer sheath configured to facilitate entry of the aspiration catheter 110 to the patient e.g., at the femoral artery. Alternatively, or additionally, in some embodiments of the disclosure, the catheter device 102 may include a guide sheath or guide catheter having a sufficient length extended to the cerebral anatomy, configured to support the aspiration catheter 110 or other interventional devices. The introducer sheath and/or guide sheath may be coupled to the hub 106 via a valve such as an RHV. Further, the catheter device 102 may include a push/pull element coupled to the aspiration catheter 110. The push/pull element may be controlled e.g., by a control button on the interface unit 106. Operation of the push/pull element may produce an axial movement of the aspiration catheter 110 in the proximal or distal direction. Alternatively, the proximal end of a push/pull element may exit through a port on the hub 106 to allow the user to manually grasp and push or pull, to thereby move the aspiration catheter 110 in the proximal or distal direction.

With reference now to FIG. 2, an enlarged cross-sectional view of a portion of an example aspiration catheter 110 according to embodiments of the disclosure is shown. The aspiration catheter 110 comprises an elongate tubular body 112 having a proximal end portion 116 and a distal end portion 118. The elongate tubular body 112 may comprise an inner liner 120 or 220 and one or more outer layers 122, 124 over the inner liner 120 or 220. The inner liner 120 or 220 may extend through the elongate tubular body 112 defining a lumen 114 of the elongate tubular body 112. The one or more outer layers 122, 124 may provide the aspiration catheter 110 with desirable properties as will be described in greater detail below.

The inner liner 120 or 220 can be constructed from a lubricious or low-friction material to provide a smooth surface for advancement of devices or objects through the inner lumen. Suitable lubricious materials include but are not limited to polytetrafluoroethylene (PTFE), fluorinated ethylene propylene (FEP), and other suitable polymeric materials.

The one or more outer layers 122, 124 may include a jacket or sheath layer 122 to provide mechanical integrity to the aspiration catheter 110. The jacket layer 122 may be constructed from a material such as a thermoplastic elastomer (TPE) e.g., polyether block amide, thermoplastic polyurethane, polyethylene, nylon, or the like. The jacket layer 122 may extend from the proximal end portion 116 to the distal end portion 118 of the elongate tubular body 112.

The one or more outer layers 122, 124 may also include a reinforcement layer 124 incorporated between the inner liner 120 or 220 and the jacket layer 122. The reinforcement layer 124 can be constructed to prevent kinking or flattening of the inner lumen 114 of the elongate tubular body 112 in navigation through a tortuous vasculature. The reinforcement layer 124 can be made from metal such as stainless steel, nitinol, or from polymers, or any combinations thereof. The reinforcement layer 124 can be in a structure of a coil or braid, or flexible tubing with a laser-cut pattern, or a hypotube such as a cut nitinol hypotube or cut rigid polymer, or the like. The reinforcement layer 124 may extend from the proximal end portion 116 to the distal end portion 118 of the elongate tubular body 112, or substantially entire length of the elongate tubular body 112. Alternatively, the reinforcement layer 124 may extend partially along the elongate tubular body 112. For example, the reinforcement layer 124 may be omitted in a portion of the proximal end portion 116 of the elongate tubular body 112.

The inner liner 120 or 220 and the one or more outer layers 122, 124 may be configured to provide the catheter 110 with varying flexibilities, rigidity, hardness, and/or other properties in/along the elongate tubular body 112. For example, the inner liner 102 or 220 and the one or more outer layers 122, 124 may be configured to render the proximal end portion 116 of the catheter 110 harder or more rigid to improve the pushability of the aspiration catheter 110, and/or render the distal end portion 118 of the catheter 110 more flexible to facilitate navigating through a tortuous vasculature.

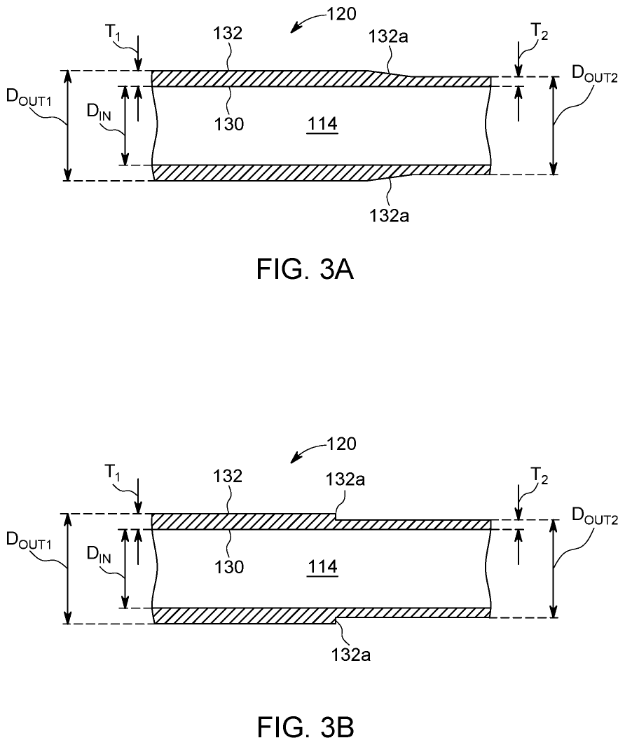

With reference to FIGS. 3A-3B and FIG. 2, enlarged cross-sectional views of a portion of an example inner liner 120 according to embodiments of the disclosure are shown. The inner liner 120 may have a variable thickness along the elongate tubular body 112 of the catheter 110. For example, the inner liner 120 may have a first thickness T₁ at the proximal end portion 116 of the elongate tubular body 112 and a second thickness T₂ at the distal end portion 118 of the elongate tubular body 112. The second thickness T₂ may be smaller than the first thickness T₁. By way of example, the first thickness T₁ may range from 0.0005 to 0.001 inches. The second thickness T₂ may range from 0.0003 to 0.0009 inches. As described above, the inner liner 120 of the aspiration catheter 110 can be constructed from a lubricious or low-friction material such as PTFE or PEP, which is generally stiff. By varying the thickness of the inner liner 120, e.g., reducing the thickness of PTFE at the distal end portion 118 of the elongate tubular body 112, the flexibility of the distal end portion 118 of the aspiration catheter 110 can be improved substantially.

The thickness of the inner liner 120 can be varied by removing materials from selected portions of an inner liner using various means including mechanical means, thermal means, electrical means, chemical means, or any combination thereof. For example, a distal end portion of an inner liner can be thinned via an abrasive process using a grinding workpiece, or by cutting using a micro-cutting tool. As another example, the material of an inner liner can be selectively removed via electrical discharge machining (EMD) using a series of rapidly recurring current discharges between electrodes. The material of an inner liner can also be selectively removed by immersing e.g., a distal end portion of the inner liner in a chemical solution. Alternatively, or additionally, the thickness of an inner liner can be varied or controlled by selective deposition of liner materials. For example, using a mandrel, a thickness of the inner liner can be built up and at some point, deposition of the liner material can be discontinued at a distal end portion of the inner liner, but continued at a proximal end portion of the inner liner.

With reference to FIGS. 3A-3B and FIG. 2, the inner liner 120 may comprise a first surface 130 and a second surface 132, wherein the first surface 130 defines the lumen 114 of the elongate tubular body 112. According to embodiments of the disclosure, the second surface 132 of the inner liner 120 defines a first outer diameter D_{OUT1} at the proximal end portion 116 of the elongate tubular body 112 and a second outer diameter D_{OUT2} at the distal end portion 118 of the elongate tubular body 112, wherein the second outer diameter D_{OUT2} is smaller than the first outer diameter D_{OUT1}. By way of example, the first outer diameter D_{OUT1} defined by the second surface 132 of the inner liner 120 at the proximal end portion 116 may range from 0.056 to 0.106 inches, and the second outer diameter D_{OUT2} defined by the second surface 132 of the inner liner 120 at the distal end portion 118 may range from 0.053 to 0.102 inches.

With reference to FIGS. 3A-3B and FIG. 2, according to embodiments of the disclosure, the second surface 132 of the inner liner 120 may comprise a transition section 132a between the proximal end portion 116 and the distal end portion 118 of the elongate tubular body 112. The transition section 132a may be tapered in the distal direction as shown in FIG. 3A. Alternatively, the transition section 132a may include a step function e.g., a vertical step as shown in FIG. 3B. Proximal to the transition section 132a, the first outer diameter D_{OUT1} defined by the second surface 132 of the inner liner 120 may be substantially constant. Distal to the transition section 132a, the second outer diameter D_{OUT2} defined by the second surface 132 of the inner liner 120 may be substantially constant.

With reference to FIGS. 3A-3B and FIG. 2, according to various embodiments of the disclosure, while the second surface 132 of the inner liner 120 may define a variable outer diameter along the length of the elongate tubular body 112, the first surface 130 of the inner liner 120 may define a lumen 114 having a substantially constant diameter D_{IN} of the elongate tubular body 112. Alternatively, the first surface 132 of the inner liner 120 may define a lumen having a variable diameter.

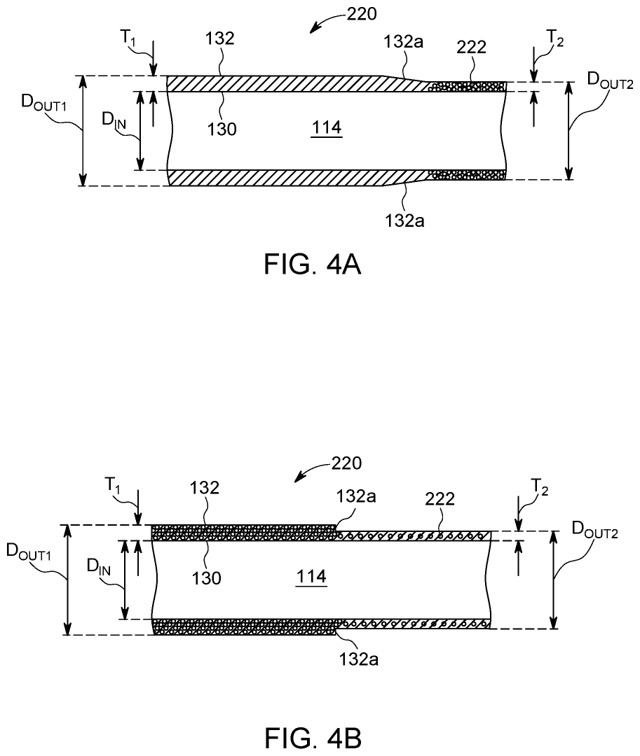

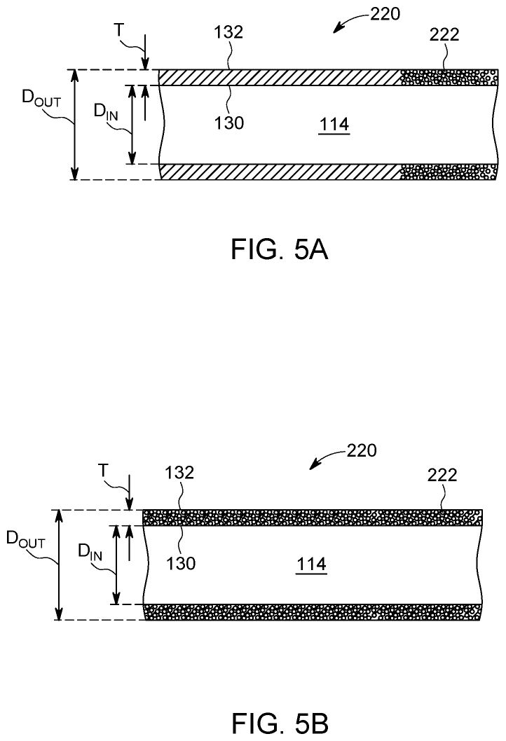

With reference to FIGS. 4A-4B, 5A-5B, and FIG. 2, enlarged cross-sectional views of a portion of an example inner liner 220 according to embodiments of the disclosure are shown. The inner liner 220 may be provided with plural voids or bubbles 222 between the first surface 130 and the second surface 132 of the inner liner 220. The plural voids or bubbles 222 may be provided at the distal end portion 118 of the elongate tubular body 112, as shown in FIGS. 4A and 5A. Alternatively, a plural voids or bubbles 222 may be provided at both the distal end portion 118 and the proximal end portion 116 of the elongate tubular body 112, or along the entire length or substantially entire length of the elongate tubular body 112, as shown in FIGS. 4B and 5B. As described above, the inner liner 220 of the aspiration catheter 110 can be constructed from a lubricious or low-friction material such as PTFE or PEP, which generally is stiff. By creating voids or bubbles 222 in the inner liner 220, the stiff liner material is replaced with empty space, resulting in the same effect as thinning the liner thickness. As such, the flexibility and trackability of the catheter 220 can be improved.

The bubbles or voids 222 can be created in the inner liner 220 using various methods known in the art, including e.g., ultrasound extraction, flow boiling and condensation, aeration, bubble nucleation, mechanical agitation, extrusion, and so on. By way of example, in an extrusion method, a dispersion of e.g., a polymeric material may pass a commercially available mini-extruder through a porous membrane to arrive at a dispersion of monodispersed vesicles. Using the extrusion technique, nanobubbles may be uniformly formed in e.g., a PTFE layer. The various techniques of forming bubbles or microbubbles are generally known in the art, and thus their detailed description is omitted herein to avoid obscuring the description of embodiments of the disclosure.

With reference to FIGS. 4A-4B and FIG. 2, the inner liner 220 may comprise a first surface 130 and a second surface 132, wherein the first surface 130 defines the lumen 114 of the elongate tubular body 112. According to embodiments of the disclosure, the second surface 132 of the inner liner 220 defines a first outer diameter D_{OUT1} at the proximal end portion 116 of the elongate tubular body 112 and a second outer diameter D_{OUT2} at the distal end portion 118 of the elongate tubular body 112, wherein the second outer diameter D_{OUT2} is smaller than the first outer diameter D_{OUT1}. Thus, the inner liner 220 may be thicker at the proximal end portion 116 of the elongate tubular body 112 and thinner at the distal end portion 118 of the elongate tubular body 112. The plural voids or bubbles 222 can be formed only at the thinner distal end portion 118 of the elongate tubular body 112, as shown in FIG. 4A. Alternatively, the plural voids or bubbles 222 can be formed at both the thinner distal end portion 118 and the thicker proximal end portion 116 of the elongate tubular body 112, or along the entire length or substantially entire length of the elongate tubular body 112, as shown in FIG. 4B.

With reference to FIGS. 4A-4B and FIG. 2, according to embodiments of the disclosure, the second surface 132 of the inner liner 220 may comprise a transition section 132a between the proximal end portion 116 and the distal end portion 118 of the elongate tubular body 112. The transition section 132a may be tapered in the distal direction as shown in FIG. 4A, or has a step function e.g., a vertical step as shown in FIG. 4B. Proximal to the transition section 132a, the first outer diameter D_{OUT1} defined by the second surface 132 of the inner liner 220 may be substantially constant. Distal to the transition section 132a, the second outer diameter D_{OUT2} defined by the second surface 132 of the inner liner 220 may be substantially constant.

With reference to FIGS. 4A-4B and FIG. 2, according to various embodiments of the disclosure, the first surface 130 of the inner liner 220 may define a lumen 114 having a substantially constant diameter D_{IN} of the elongate tubular body 112. Alternatively, the first surface 130 of the inner liner 220 may define a lumen having a variable diameter.

With reference to FIGS. 5A-5B and FIG. 2, enlarged cross-sectional views of a portion of an example inner liner 220 according to alternative embodiments of the disclosure are shown. The inner liner 220 may comprise a first surface 130 and a second surface 132, wherein the first surface 130 defines the lumen 114 of the elongate tubular body 112. According to embodiments of the disclosure, the second surface 132 of the inner liner 220 may define a substantially constant outer diameter D_{OUT} along the entire length or substantially entire length of the inner liner 220. Thus, the inner liner 220 may have a substantially constant thickness T along the entire length of the elongate tubular body 112. The plural voids or bubbles 222 can be formed in the inner liner 220 only at the distal end portion 118 of the elongate tubular body 220, as shown in FIG. 5A. Alternatively, the plural voids or bubbles 220 can be formed in the inner liner 220 at both the distal end portion 116 and the proximal end portion 118 of the elongate tubular body 112, or along the entire length or substantially entire length of the elongate tubular body 112, as shown in FIG. 5B.

With reference to FIGS. 5A-5B, according to various embodiments of the disclosure, the first surface 130 of the inner liner 220 may define a lumen 114 having a substantially constant diameter D_{IN} along the length of the elongate tubular body 112. Alternatively, the first surface 132 of the inner liner 220 may define a lumen 114 having a variable diameter along the length of the elongate tubular body.

Returning to FIG. 1, the size and/or dimension of the elongate tubular body 112 of the aspiration catheter 110 depends on the medical application and access site. By way of example, for neurovascular applications via femoral artery access, the elongate tubular body 112 may have a length ranging from about 105 cm to about 150 cm or more. The outer diameter of the elongate tubular body may range from about 1.17 mm to about 2.74 mm. The inner diameter of the lumen of the elongate tubular body may range from about 0.92 mm to about mm 2.39. It should be noted that the specific dimensions and sizes are provided by way of example for understanding embodiments of the disclosure. The appended claims are not limited to the specific dimensions and sizes.

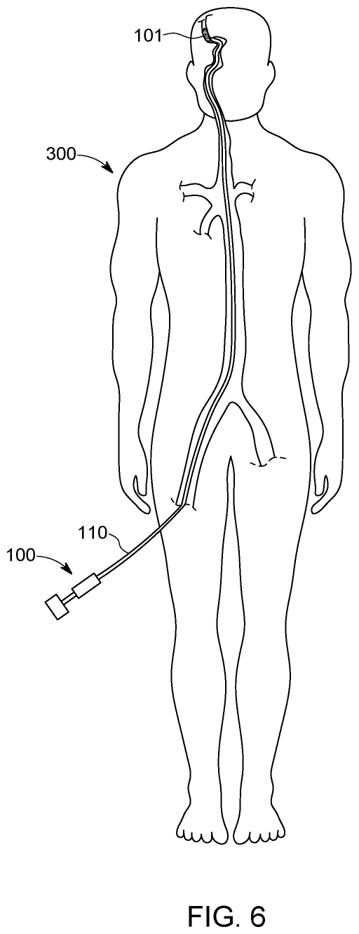

FIG. 6 depicts an example aspiration system 100 of the disclosure used for treating a patient 300 in a thrombectomy procedure to remove an occlusion 101 within the vasculature at the cerebral anatomy. The aspiration catheter 110 may access the treatment site at the cerebral anatomy through entry e.g., at the femoral artery of the patient 300 by using an introducer sheath. The aspiration catheter 110 may be transluminally navigated along or over a guidewire to the treatment site. In some embodiment, the catheter device may include a guide catheter which can be introduced to the vasculature in the cerebral anatomy. The aspiration catheter 110 within the guide catheter can be distally navigated to allow the distal end portion of the aspiration catheter to reach the occlusion 101. The occlusion 101 can be then drawn into the distal end portion of the aspiration catheter upon application of a negative pressure provided by the vacuum source. Once the occlusion 101 has been drawn into the distal end portion of the aspiration catheter, it may be proximally withdrawn from the body. The aspiration catheter 110 can be thereafter proximally withdrawn from the body.

Various embodiments of an aspiration catheter, system, and method have been described with reference to figures. It should be noted that the figures are intended for illustration of embodiments but not for exhaustive description or limitation on the scope of the disclosure. Alternative structures, components, and materials will be readily recognized as being viable without departing from the principle of the claimed invention.

All technical and scientific terms used herein have the meaning as commonly understood by one of ordinary skill in the art unless specifically defined otherwise. As used in the description and appended claims, the singular forms of “a,” “an,” and “the” include plural references unless the context clearly dictates otherwise. The term “or” refers to a nonexclusive “or” unless the context clearly dictates otherwise. The term “proximal” and its grammatically equivalent refers to a position, direction or orientation towards the user or physician's side. The term “distal” and its grammatically equivalent refers to a position, direction or orientation away from the user or physician's side. The term “first” or “second” etc. may be used to distinguish one element from another in describing various similar elements. It should be noted the terms “first” and “second” as used herein include references to two or more than two. Further, the use of the term “first” or “second” should not be construed as in any particular order unless the context clearly dictates otherwise.

Those skilled in the art will appreciate that various other modifications may be made. All these or other variations and modifications are contemplated by the inventors and within the scope of the invention.

What is claimed is:

1. A catheter comprising an elongate tubular body having a proximal end portion and a distal end portion, the elongate tubular body comprising:
an inner liner comprising a first surface and a second surface, the first surface defining a lumen of the elongate tubular body; and
one or more outer layers over the second surface of the inner liner,
wherein the inner liner comprises a first thickness at the proximal end portion of the elongate tubular body and a second thickness at the distal end portion of the elongate tubular body, the second thickness being smaller than the first thickness.

2. The catheter of claim 1, wherein the second surface of the inner liner defines a first outer diameter at the proximal end portion of the elongate tubular body and a second outer diameter at the distal end portion of the elongate tubular body, the second outer diameter being smaller than the first outer diameter.

3. The catheter of claim 2, wherein the first outer diameter defined by the second surface of the inner liner at the proximal end portion is substantially constant, the second outer diameter defined by the second surface of the inner liner at the distal end portion is substantially constant, and the second surface of the inner liner further comprises a transition section having a tapering function from the first outer diameter to the second outer diameter.

4. The catheter of claim 2, wherein the first outer diameter defined by the second surface of the inner liner at the proximal end portion is substantially constant, the second outer diameter defined by the second surface of the inner liner at the distal end portion is substantially constant, and the second surface of the inner liner further comprises a transition section having a step function from the first outer diameter to the second outer diameter.

5. The catheter of claim 2, wherein the first surface of the inner liner defines a diameter of the lumen substantially constant between the proximal end portion and the distal end portion of the elongate tubular body.

6. The catheter of claim 2, wherein the inner liner is constructed from a material comprising polytetrafluoroethylene (PTFE).

7. The catheter of claim 6, wherein the first outer diameter defined by the second surface of the inner liner at the proximal end portion is substantially constant, the second outer diameter defined by the second surface of the inner liner at the distal end portion is substantially constant, and the second surface of the inner liner further comprises a transition section having a tapering function from the first outer diameter to the second outer diameter.

8. The catheter of claim 1, wherein the inner liner is provided with plural voids between the first surface and the second surface of the inner liner.

9. The catheter of claim 8A, wherein the plural voids are located at the distal end portion of the elongate tubular body.

10. The catheter of claim 8A, wherein the plural voids are located throughout the entire length of the elongate tubular body from the proximal end portion to the distal end portion.

11. A catheter comprising an elongate tubular body having a proximal end portion and a distal end portion, the elongate tubular body comprising:
an inner liner comprising a first surface and a second surface, the first surface defining a lumen of the elongate tubular body; and
one or more outer layers over the second surface of the inner liner,
wherein the inner liner is provided with plural voids between the first surface and the second surface of the inner liner.

12. The catheter of claim 11, wherein the plural voids are located at the distal end portion of the elongate tubular body.

13. The catheter of claim 11, wherein the plural voids are located throughout the entire length of the elongate tubular body from the proximal end portion to the distal end portion.

14. The catheter of claim 11, wherein the inner liner comprises a first thickness at the proximal end portion of the elongate tubular body and a second thickness at the distal end portion of the elongate tubular body, the second thickness being smaller than the first thickness.

15. The catheter of claim 11, wherein the second surface of the inner liner defines a first outer diameter at the proximal end portion of the elongate tubular body and a second outer diameter at the distal end portion of the elongate tubular body, the second outer diameter being smaller than the first outer diameter.

16. The catheter of claim 15, wherein the first outer diameter defined by the second surface of the inner liner at the proximal end portion is substantially constant, the second outer diameter defined by the second surface of the inner liner at the distal end portion is substantially constant, and the second surface of the inner liner further comprises a transition section having a tapering function from the first outer diameter to the second outer diameter.

17. The catheter of claim 11, wherein the inner liner is constructed from a material comprising polytetrafluoroethylene (PTFE).

18. The catheter of claim 11, wherein the elongate tubular body comprises a length between the proximal end portion and the distal end portion, and the inner liner extends the length of the elongate tubular body and comprises a substantially constant thickness along the length.

19. The catheter of claim 18, wherein the plural voids are located at the distal end portion of the elongate tubular body.

20. The catheter of claim 18, wherein the plural voids are located throughout the entire length of the elongate tubular body.
